(19) United States
(12) Patent Application Publication
(10) Pub. No.: US 2018/0104161 A1
(43) Pub. Date: Apr. 19, 2018

(54) METHOD OF USING A COSMETIC MASCARA AND FIBER

(71) Applicant: YOUNIQUE, LLC, Lehi, UT (US)

(72) Inventor: Mukhtar Siddiqui, Lehi, UT (US)

(73) Assignee: YOUNIQUE, LLC, Lehi, UT (US)

(21) Appl. No.: 15/564,699

(22) PCT Filed: Apr. 7, 2015
(86) PCT No.: PCT/US2015/024768

§ 371 Date: Oct. 5, 2017

(51) Int. Cl. A61K 8/02; A61K 8/04; A61Q 1/10; A45D 34/04; A41G 5/02

(52) U.S. Cl. CPC A61K 8/027; A61K 8/042; A41G 5/02; A45D 34/042; A61Q 1/10

(57) ABSTRACT

The present disclosure describes and enables a method of lengthening the appearance of a subject's eye lashes through use of a cellulosic fiber in combination with transplanting gel and a system therefor. The disclosed process and system allows a user to lengthen their eyelashes through use of an only semisynthetic naturally derived fiber such as rayon, which allows a user to avoid use of a fully synthetic fiber such as nylon. The method of the present disclosure comprises applying mascara to a subject's eyelashes and then applying the fibers with a brush while the mascara is still wet. In some cases a transplanting gel is applied first (which may comprise mascara) and then mascara can be applied after the fibers have been applied to the subject's eyelashes. In additional cases, the fibers have also been treated prior to use.

### FIELD

The present disclosure is in the field of cosmetics.

### BACKGROUND

Enhancing the beauty of one's eyes is of significant importance to consumers and has led to the development of very large product categories in both cosmetics and skin care. In particular, enhancing the visibility of eyelashes has become a large industry that continues to grow.

Mascara is used to enhance the beauty of a person's eyes by coating the lashes and causing them to appear thicker, longer, and/or darker. There are two types of mascaras used to enhance the thickness and length of eye lashes. The first type is a gel with black pigment and second type is a gel with nylon or other types of fiber dispersed within the gel. For example, U.S. Pat. No. 7,754,196 describes a mascara comprising rigid, substantially rectilinear fibers of a synthetic polymer mixed into a physiologically acceptable medium and applied as a single mascara to a subject's eyelashes.

There are also mascara systems such as “Better than False Lashes” from Too Faced that involve a two-step process wherein 1-2 coats of mascara are applied first to create a base to adhere nylon fibers. In a second step nylon fibers are applied to the subject's lashes and then the lashes are coated with mascara again. The fiber and mascara coating system results in an extended lash look which subjects desire.

Some problems with the use of nylon fibers include that the fibers are synthetic and non-biodegradable. The current innovation overcomes these problems.

### SUMMARY

The present disclosure describes and enables a method of lengthening the appearance of a subject's eye lashes through use of a cellulosic fiber in combination with mascara and a system therefor. The disclosed process and system allows a user/subject to lengthen their eyelashes through use of an only semisynthetic naturally derived fiber, which allows a user to avoid use of a fully synthetic fiber such as nylon. The method of the present disclosure comprises applying a transplanting gel (which may be a mascara) to a subject's eyelashes and then applying the fibers with a brush while the transplanting gel/mascara is still wet. The transplanting gel/mascara catches and at least partially coats the fibers adhering them to the subject's eye lashes and causes the eyelashes to appear lengthened.

The present disclosure also describes treating the fibers, such as with natural extracts, for a variety of purposes, such as conditioning the fibers and/or coloring the fibers. This provides an advantage in that the fibers can be color matched to the transplanting gel/mascara to create a better eyelash coloration match from the combination of the transplanting gel/mascara and fibers.

The foregoing and other objects, features, and advantages of this disclosure will become more apparent from the following detailed description.

### DETAILED DESCRIPTION

The present disclosure relates to a method of using a system of fibers and transplanting gel/mascara to lengthen the appearance of a subject's eyelashes. Briefly, this disclosure describes applying mascara to a subject's eyelashes, in optional cases applying a transplanting gel (which could also be applied as an alternative to the mascara), and then applying rayon fibers to the eyelashes to lengthen the appearance of the subject's eyelashes. In some cases, mascara is also applied after the fibers are applied.

Mascara in this disclosure comprises a non-toxic medium that is compatible with human keratin materials, especially the eyelashes, for example a cosmetic medium, the cosmetic medium possibly being a hydrophilic or lipophilic cosmetic medium.

The mascara according to the disclosure may comprise an aqueous medium, constituting an aqueous phase, which may be the continuous phase of the mascara.

The mascara may comprise water and optionally one or more hydrophilic organic solvent(s), i.e. one or more water-miscible organic solvent(s), for instance alcohols and especially monoalcohols containing from 2 to 5 carbon atoms, for instance ethanol, isopropanol or n-propanol, polyols containing from 2 to 8 carbon atoms, for instance glycerol, diglycerol, propylene glycol, ethylene glycol, 1,3-butylene glycol, sorbitol, pentylene glycol, C₃-C₄ ketones and C₂-C₄ aldehydes.

The water or the mixture of water and hydrophilic organic solvent(s) may be present in the mascara according to the disclosure in a content ranging from 0.1% to 90% by weight and preferably from 0.1% to 60% by weight relative to the total weight of the mascara.

The mascara may also comprise a fatty phase, which may comprise fatty substances chosen from oils, organic solvents, waxes and pasty fatty substances, and mixtures thereof. The fatty phase may form a continuous phase of the mascara. In particular, the mascara according to the disclosure may be anhydrous.

The fatty phase may consist especially of any physiologically acceptable and in particular cosmetically acceptable oil, chosen especially from carbon-based oils, hydrocarbon-based oils, fluoro oils and/or silicone oils of mineral, animal, plant or synthetic origin, alone or as a mixture.

The total fatty phase of the mascara may represent from 0.1% to 98% by weight and preferably from 1% to 80% by weight relative to the total weight of the mascara.

Advantageously, the fatty phase of the mascara may comprise at least one volatile organic solvent or oil and/or at least one non-volatile oil. More preferably, the fatty phase comprises at least one volatile oil.

For the purposes of the disclosure, the expression “volatile compound”, for example “volatile oil or organic solvent”, means any compound (or non-aqueous medium) that can evaporate on contact with the skin or the keratin fiber or material in less than one hour at room temperature and atmospheric pressure. The volatile compound, for example the volatile organic solvent(s) and the volatile oils of the disclosure, is a volatile cosmetic compound (these are, for example, organic solvents and volatile cosmetic oils), which is liquid at room temperature, especially having a nonzero vapour pressure at room temperature and atmospheric pressure, ranging in particular from 10⁻³ to 300 mmHg (0.13 Pa to 40 000 Pa), more particularly ranging from 1.3 Pa to 13 000 Pa (0.01 to 100 mmHg) and even more particularly ranging from 1.3 Pa to 1 300 Pa (0.01 to 10 mmHg). In contrast, the term “non-volatile compound”, for example “non-volatile oil”, means a compound for example an oil that remains on the skin or the keratin fiber or material at room temperature and atmospheric pressure for at least several hours and that especially has a vapour pressure of less than 10³ mmHg (0.13 Pa).

These oils may be hydrocarbon-based oils, silicone oils or fluorinated oils, or mixtures thereof.

The term “hydrocarbon-based oil” means an oil mainly containing hydrogen and carbon atoms and optionally oxygen, nitrogen, sulphur or phosphorus atoms. The volatile hydrocarbon-based oils may be chosen from hydrocarbon-based oils containing from 8 to 16 carbon atoms, and especially branched C₈-C₁₆ alkanes, for instance C₈-C₆ isoalkanes of petroleum origin (also known as isoparaffins), for instance isododecane (also known as 2,2,4,4,6-pentamethylheptane), isodecane and isohexadecane, for example the oils sold under the trade names Isopar or Permetyl, branched C₈-C₁₆ esters and isohexyl neopentanoate, and mixtures thereof. Other volatile hydrocarbon-based oils, for instance petroleum distillates, especially those sold under the name Shell Solt by the company Shell, may also be used. The volatile solvent is preferably chosen from volatile hydrocarbon-based oils containing from 8 to 16 carbon atoms, and mixtures thereof.

Volatile oils that may also be used include volatile silicones, for instance volatile linear or cyclic silicone oils, especially those with a viscosity ≤6 centistokes (6×10⁻⁶ m²/s) and especially containing from 2 to 10 silicon atoms, these silicones optionally comprising alkyl or alkoxy groups containing from 1 to 22 carbon atoms. These may be made of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane and dodecamethyl pentasiloxane, and mixtures thereof.

Volatile fluorinated solvents such as nonafluoromethoxybutane or perfluoromethylcyclopentane may also be used.

The volatile oil may be present in the mascara according to the disclosure in a content ranging from 0.1% to 98% by weight and preferably from 1% to 65% by weight relative to the total weight of the mascara.

The mascara may also comprise at least one non-volatile oil chosen especially from non-volatile hydrocarbon-based oils and/or silicone and/or fluorinated oils.

Non-volatile hydrocarbon-based oils that may include:
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, karite oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel,
synthetic ethers containing from 10 to 40 carbon atoms;
linear or branched hydrocarbons of mineral or synthetic origin, such as petroleum jelly, polydecenes, hydrogenated polyisobutene such as parleam, and squalane, and mixtures thereof;
synthetic esters such as oils of formula R₁COOR₂ in which R₁ represents a linear or branched fatty acid residue containing from 1 to 40 carbon atoms and R₂ represents an in particular branched hydrocarbon-based chain containing from 1 to 40 carbon atoms, on condition that R₅+R₆≥10, such as, for example, purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
higher fatty acids such as oleic acid, linoleic acid or linolenic acid;
and mixtures thereof.

The non-volatile silicone oils that may be used in the mascara according to the disclosure may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenylsilicones, for instance phenyltrimethicones, phenyldimethicones, phenyltrimethylsiloxydiphenylsiloxanes, diphenyldimethicones, diphenylmethyldiphenyltrisiloxanes and 2-phenylethyl-trimethylsiloxysilicates.

The fluorinated oils that may be used in the disclosure are, in particular, fluorosilicone oils, fluorinated polyethers or fluorinated silicones, as described in document EP-A-847 752.

The non-volatile oils may be present in the mascara according to the disclosure in a content ranging from 0.1% to 80% by weight, preferably from 0.1% to 50% by weight and better still from 0.1% to 20% by weight, relative to the total weight of the mascara.

The fatty phase of the mascara according to the disclosure may comprise a wax. For the purposes of the present disclosure, the term “wax” means a lipophilic fatty compound that is solid at room temperature (25° C.) and atmospheric pressure (760 mmHg, i.e. 10⁵ Pa), with a reversible solid/liquid change of state, having a melting point of greater than 30° C. and better still greater than 55° C., which may be up to 200° C. and especially up to 120° C.

By bringing the wax to its melting point, it is possible to make it miscible with oils and to form a microscopically homogeneous mixture, but on cooling the mixture to room temperature, recrystallization of the wax in the oils of the mixture is obtained.

The melting point values correspond, according to the disclosure, to the melting peak measured using a differential scanning calorimeter (D.S.C.), for example the calorimeter sold under the name DSC 30 by the company Mettler, with a temperature rise of 5 or 10° C. per minute.

For the purposes of the disclosure, the waxes are those generally used in cosmetics and dermatology. These may include beeswax, lanolin wax and Chinese insect waxes; rice wax, carnauba wax, candelilla wax, ouricury wax, cork fiber wax, sugar cane wax, Japan wax and sumach wax; montan wax, microcrystalline waxes, paraffin waxes, ozokerites, ceresin wax, lignite wax, polyethylene waxes, the waxes obtained by Fischer-Tropsch synthesis, and fatty acid esters of glycerides that are solids at 40° C. and better still at more than 55° C.

These may further include waxes obtained by catalytic hydrogenation of animal or plant oils containing linear or branched C₈-C₃₂ fatty chains, for example, hydrogenated jojoba oil, hydrogenated sunflower oil, hydrogenated castor oil, hydrogenated coconut oil and hydrogenated lanolin oil.

Waxes may also be made of silicone waxes or fluorinated waxes.

The waxes present in the mascara may be dispersed in the form of particles in an aqueous medium. These particles may have a mean size ranging from 50 nm to 10 μm and preferably from 50 nm to 3.5 μm.

In particular, the wax may be present in the form of a wax-in-water emulsion, the waxes possibly being in the form of particles with a mean size ranging from 1 μm to 10 μm and preferably from 1 μm to 3.5 μm.

In another embodiment of the mascara according to the disclosure, the wax may be present in the form of a wax microdispersion, the wax being in the form of particles with a mean size of less than 1 μm and ranging especially from 50 nm to 500 nm. Wax microdispersions are described in documents EP-A-557 196 and EP-A-1 048 282.

The wax may also have a hardness ranging from 0.05 MPa to 15 MPa and preferably ranging from 6 MPa to 15 MPa. The hardness is determined by measuring the compressive strength, measured at 20° C. using a texturometer sold under the name TA-XT2i by the company Rheo, equipped with a stainless-steel cylinder 2 mm in diameter travelling at a measuring speed of 0.1 mm/s, and penetrating into the wax to a penetration depth of 0.3 mm. To measure the hardness, the wax is melted at a temperature equal to the melting point of the wax +20° C. The molten wax is cast in a container 30 mm in diameter and 20 mm deep. The wax is recrystallized at room temperature (25° C.) over 24 hours and is then stored for at least 1 hour at 20° C. before performing the hardness measurement. The value of the hardness is the compressive strength measured divided by the area of the texturometer cylinder in contact with the wax.

The wax may be present in the mascara according to the disclosure in a content ranging from 0.1% to 50% by weight, preferably from 0.5% to 30% by weight and better still from 1% to 20% by weight, relative to the total weight of the mascara.

The mascara according to the disclosure may contain at least one fatty compound that is pasty at room temperature. For the purposes of the disclosure, the expression “pasty fatty substance” means fatty substances with a melting point ranging from 20 to 55° C., preferably 25 to 45° C., and/or a viscosity at 40° C. ranging from 0.1 to 40 Pa·s (1 to 400 poises), preferably 0.5 to 25 Pa·s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of his general knowledge, so as to be able to carry out the measurement of the pasty compound tested.

These fatty substances are preferably hydrocarbon-based compounds, optionally of polymeric type; they can also be chosen from silicone and/or fluorinated compounds; they may also be in the form of a mixture of hydrocarbon-based and/or silicone and/or fluorinated compounds. In the case of a mixture of different pasty fatty substances, the hydrocarbon-based pasty compounds (containing mainly hydrogen and carbon atoms and optionally ester groups) are preferably used in major proportion.

Among the pasty compounds which may be used in the mascara according to the disclosure, included are those made of lanolins and lanolin derivatives such as acetylated lanolins or oxypropylenated lanolins or isopropyl lanolate, having a viscosity of from 18 to 21 Pa·s (Pascal-second), preferably 19 to 20.5 Pa·s, and/or a melting point of from 30 to 55° C., and mixtures thereof. It is also possible to use esters of fatty acids or of fatty alcohols, in particular those containing from 20 to 65 carbon atoms (melting point of about from 20 to 35° C. and/or viscosity at 40° C. ranging from 0.1 to 40 Pa·s), such as triisostearyl or cetyl citrate; arachidyl propionate; polyvinyl laurate; cholesterol esters, such as triglycerides of plant origin, such as hydrogenated plant oils, viscous polyesters such as poly(12-hydroxystearic acid), and mixtures thereof. Triglycerides of plant origin that may be used include hydrogenated castor oil derivatives, such as “Thixinr” from Rhéox.

This may also be made of pasty silicone fatty substances such as polydimethylsiloxanes (PDMSs) containing pendent chains of the alkyl or alkoxy type containing from 8 to 24 carbon atoms, and having a melting point of 20-55° C., such as stearyldimethicones, in particular those sold by Dow Corning under the trade names DC2503 and DC25514, and mixtures thereof.

The pasty fatty substance may be present in the mascara according to the disclosure in a proportion of from 0% to 60% (especially 0.01% to 60%) by weight, relative to the total weight of the mascara, preferably in a proportion of from 0.5% to 45% by weight, and better still ranging from 2% to 30% by weight, in the mascara.

The mascara according to the disclosure can contain emulsifying surfactants, present in particular in a proportion or from 5% to 15%. These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of the said reference, for the anionic and nonionic surfactants.

The surfactants preferably used in the mascara according to the disclosure are chosen from:
nonionic surfactants: such as, fatty acids, fatty alcohols, polyethoxylated or polyglycerolated fatty alcohols such as polyethoxylated stearyl or cetylstearyl alcohols, fatty acid esters of sucrose, alkyl glucose esters, in particular polyoxyethylenated fatty esters of C₁-C₆ alkyl glucose, and mixtures thereof;
anionic surfactants: such as, C₁₆-C₃₀ fatty acids neutralized with amines, aqueous ammonia or alkaline salts, and mixtures thereof.

Surfactants which make it possible to obtain an oil-in-water or wax-in-water emulsion are preferably used.

The mascara according to the disclosure may comprise a film-forming polymer.

The film-forming polymer may be a polymer that is dissolved or dispersed in the form of particles in an aqueous phase of the mascara, or dissolved or dispersed in the form of particles in a liquid fatty phase. The mascara may comprise a blend of these polymers.

The film-forming polymer may be present in the mascara according to the disclosure in a solids content ranging from 0.1% to 60% by weight, preferably from 0.5% to 40% by weight and better still from 1% to 30% by weight, relative to the total weight of the mascara.

In the present application, the expression “film-forming polymer” means a polymer which is capable, by itself or in the presence of an auxiliary film-forming agent, of forming a continuous and adherent film on a support, in particular on keratin materials.

A film-forming polymer capable of forming a hydrophobic film, i.e. a polymer whose film has a solubility in water at 25° C. of less than 1% by weight, is preferably used.

Among the film-forming polymers which may be used in the mascara of the present disclosure, these may be made of synthetic polymers, of radical-mediated type or of polycondensate type, and polymers of natural origin, and mixtures thereof.

The expression “radical-mediated film-forming polymer” means a polymer obtained by polymerization of monomers containing unsaturation, in particular ethylenic unsaturation, each monomer being capable of homopolymerizing (unlike polycondensates).

The film-forming polymers of radical-mediated type may be, in particular, vinyl polymers or copolymers, in particular acrylic polymers.

The vinyl film-forming polymers can result from the polymerization of monomers containing ethylenic unsaturation and containing at least one acidic group and/or esters of these acidic monomers and/or amides of these acidic monomers.

Monomers bearing an acidic group which may be used are α,β-ethylenic unsaturated. carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), especially (meth)acrylates of an alkyl, in particular of a C₁-C₃₀ and preferably C₁-C₂₀ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl (meth)acrylates are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates which are hydroxyethyl acrylate, 2-hydroxypropyl acrylate, hydroxyethyl methacrylate and 2-hydroxypropyl methacrylate.

Among the aryl (meth)acrylates are benzyl acrylate and phenyl acrylate.

The (meth)acrylic acid esters that are useful are the alkyl (meth)acrylates.

According to the present disclosure, the alkyl group of the esters may be either fluorinated or perfluorinated, i.e. some or all of the hydrogen atoms of the alkyl group are substituted with fluorine atoms.

Examples of amides of the acid monomers are (meth)acrylamides, and especially N-alkyl(meth)acrylamides, in particular of a C₂-C₁₂ alkyl. Among the N-alkyl(meth)acrylamides are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned above.

Examples of vinyl esters are vinyl acetate, vinyl neodecanoate, vinyl pivalate, vinyl benzoate and vinyl t-butylbenzoate.

Styrene monomers include styrene and a-methylstyrene.

It is possible to use any monomer known to the person skilled in the art that falls with the categories of acrylic monomers and vinyl monomers (including monomers modified by a silicone chain).

Among the film-forming polycondensates are polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethane-polyvinyl-pyrrolidones, polyester-polyurethanes, polyether-polyurethanes, polyureas and polyurea/polyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. Examples of such acids are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol.

Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines which may be used are ethylenediamine, hexamethylenediamine and meta- or para-phenylenediamine. An amino alcohol which may be used is monoethanolamine.

The polyester may also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group—SO₃M may be used in particular.

The aromatic nucleus of the difunctional aromatic monomer also bearing a group —SO₃M as described above may be chosen, for example, from benzene, naphthalene, anthracene, biphenyl, oxybiphenyl, sulphonylbiphenyl and methylenebiphenyl nuclei. As examples of difunctional aromatic monomers also bearing a group —SO₃M, noted are: sulphoisophthalic acid, sulphoterephthalic acid, sulphophthalic acid, 4-sulphonaphthalene-2,7-dicarboxylic acid.

The copolymers preferably used are those based on isophthalate/sulphoisophthalate, and more particularly copolymers obtained by condensation of diethylene glycol, cyclohexanedimethanol, isophthalic acid and sulphoisophthalic acid. Such polymers are sold, for example, under the brand name Eastman AQ® by the company Eastman Chemical Products.

The optionally modified polymers of natural origin may be chosen from shellac resin, sandarac gum, dammar resins, elemi gums, copal resins and cellulose-based polymers, and mixtures thereof.

According to an embodiment of the mascara according to the disclosure, the film-forming polymer may be present in the form of particles in aqueous dispersion, generally known as a latex or pseudolatex. The techniques for preparing these dispersions are well known to the person skilled in the art.

Aqueous dispersions of film-forming polymers which may be used are the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins,

Dow Latex 432® by the company Dow Chemical, Daitosol 5000 AD® by the company Daito Kasey Kogyo; or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer.

Aqueous dispersions of film-forming polymer that may also be used include polymer dispersions resulting from the free-radical polymerization of one or more radical monomers within and/or partially at the surface of pre-existing particles of at least one polymer chosen from the group consisting of polyurethanes, polyureas, polyesters, polyesteramides and/or alkyds. These polymers are generally known as hybrid polymers.

According to an embodiment of the mascara according to the disclosure, the film-forming polymer may be a water-soluble polymer and is thus present in the aqueous phase of the mascara in dissolved form. Examples of water-soluble film-forming polymers include:
proteins, for instance proteins of plant origin such as wheat proteins and soybean proteins; proteins of animal origin such as keratins, for example keratin hydrolysates and sulphonic keratins;
anionic, cationic, amphoteric or nonionic chitin or chitosan polymers;
polymers of celluloses such as hydroxyethylcellulose, hydroxypropylcellulose, methylcellulose, ethylhydroxyethylcellulose and carboxymethylcellulose, and quaternized cellulose derivatives;
acrylic polymers or copolymers, such as polyacrylates or polymethacrylates;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
polymers of natural origin, which are optionally modified, such as:
gum arabics, guar gum, xanthan derivatives, karaya gum;
alginates and carrageenans;
glycoaminoglycans, hyaluronic acid and derivatives thereof;
shellac resin, sandarac gum, dammar resins, elemi gums and copal resins;
deoxyribonucleic acid;
mucopolysaccharides such as hyaluronic acid and chondroitin sulphate, and mixtures thereof.

According to another embodiment of the mascara according to the disclosure, the film-forming polymer may be present in a liquid fatty phase comprising organic solvents or oils such as those described above. For the purposes of the disclosure, the expression “liquid fatty phase” means a fatty phase which is liquid at room temperature (25° C.) and atmospheric pressure (760 mmHg, i.e. 10⁵ Pa), composed of one or more fatty substances that are liquid at room temperature, also known as oils, which are generally mutually compatible.

The liquid fatty phase preferably comprises a volatile oil, optionally mixed with a non-volatile oil, the oils possibly being chosen from those mentioned above.

According to a further embodiment of the mascara according to the disclosure, the film-forming polymer may be present in the form of surface-stabilized particles dispersed in the liquid fatty phase.

The dispersion of surface-stabilized polymer. particles may be manufactured as described in document EP-A-749 747.

The polymer particles are surface-stabilized by means of a stabilizer that may be a block polymer, a grafted polymer and/or a random polymer, alone or as a blend.

Dispersions of film-forming polymer in the liquid fatty phase, in the presence of stabilizers, are described especially in documents EP-A-749 746, EP-A-923 928 and EP-A-930 060.

The size of the polymer particles in dispersion either in the aqueous phase or in the liquid fatty phase may range from 5 nm to 600 nm and preferably 20 nm to 300 nm.

According another embodiment of the mascara according to the disclosure, the film-forming polymer may be dissolved in the liquid fatty phase, in which case the film-forming polymer is said to be a liposoluble polymer.

Examples of liposoluble polymers are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an a-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers are the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Examples of liposoluble film-forming polymers are liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble homopolymers may be chosen from polyvinyl stearate copolymers, polyvinyl stearate copolymers crosslinked with divinylbenzene, with diallyl ether or with diallyl phthalate, polystearyl (meth)acrylate copolymers, polyvinyl laurate copolymers and polylauryl (meth)acrylate copolymers, it being possible for these poly(meth)acrylates to be crosslinked with the aid of ethylene glycol dimethacrylate or tetraethylene glycol dimethacrylate.

The liposoluble copolymers defined above are known and are described in particular in patent application FR-A-2 232 303; they may have a weight-average molecular weight ranging from 2 000 to 500 000 and preferably from 4 000 to 200 000.

Liposoluble film-forming polymers which may be used in the disclosure include polyalkylenes and in particular copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers which may be used in the disclosure are the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

The mascara according to the disclosure may comprise an auxiliary film-forming agent that promotes the formation of a film with the film-forming polymer. Such a film-forming agent may be chosen from any compound known to those skilled in the art as being capable of satisfying the desired function, and may be chosen especially from plasticizers and coalescers.

The mascara according to the disclosure may also comprise a dyestuff, for instance pulverulent dyestuffs, liposoluble dyes and water-soluble dyes. This dyestuff may be present in a content ranging from 0.01% to 30% by weight relative to the total weight of the mascara.

The pulverulent dyestuffs may be chosen from pigments and nacres.

The pigments may be white or coloured, mineral and/or organic, and coated or uncoated. Among the mineral pigments are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments are carbon black, pigments of D & C type, and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica with, in particular, ferric blue or chromium oxide, titanium mica with an organic pigment of the abovementioned type, and nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

The mascara according to the disclosure may also comprise fillers. The term “fillers” should be understood as meaning colorless or white, mineral or synthetic particles of any shape, which are insoluble in the medium of the mascara irrespective of the temperature at which the mascara is manufactured. These fillers serve especially to modify the rheology or the texture of the mascara.

The fillers may be mineral or organic and of any shape, in platelet form, spherical or oblong, irrespective of the crystallographic shape (for example leaflet, cubic, hexagonal, orthorhombic, etc.). This may include talc, mica, silica, kaolin, powders of polyamide, for instance Nylon® (Orgasol® from Atochem), of poly-β-alanine and of polyethylene, powders of tetrafluoroethylene polymers, for instance Teflon®, lauroyllysine, starch, boron nitride, hollow polymer microspheres such as those of polyvinylidene chloride/acrylonitrile, for instance Expancel® (Nobel Industrie), acrylic acid copolymers such as Polytrap® (Dow Corning) and silicone resin microbeads (for example Tospearls® from Toshiba), polyorganosiloxane elastomer particles, precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

The fillers may be present in a proportion of from 0.01% to 30% by weight and preferably 0.5% to 15% by weight.

The mascara of the disclosure may also comprise any additive usually used in cosmetics, such as antioxidants, fillers, preserving agents, fragrances, neutralizers, thickeners, surfactants, cosmetic or dermatological active agents, for instance emollients, moisturizers, vitamins, sunscreens, plasticizers and coalescers, and mixtures thereof. These additives may be present in the mascara in a content ranging from 0.01% to 20% and better still ranging from 0.01% to 10% of the total weight of the mascara.

Needless to say, a person skilled in the art will take care to select the optional additional additives and/or the amount thereof such that the advantageous properties of the mascara according to the disclosure are not, or are not substantially, adversely affected by the envisaged addition.

The mascara according to the disclosure may be manufactured by the known processes generally used in cosmetics or dermatology.

An embodiment of a mascara formulation according to this disclosure includes ingredients that are combined in various phases to form the mascara. These phases may be combined in different ways but in one embodiment there are two phases, a water phase and an oil phase. The water phase comprises water and water soluble or dispersible ingredients and may include Propylene glycol, butylene glycol, hydroxyethyl cellulose, xanthan gum, acrylates and alkyl acrylates copolymer, alcohol, allantoin, triethanolamine, aminomethyl propanol, phenoxyethanol, ethylhexylglycerine, sodium lauryl sulfate, sodium laureth sulfate, sodium dehydroacetate, panthenol, Zea Mays (corn) starch, tetrasodium EDTA, imidazodinyl urea, sodium chloride, and the oil phase comprises silicones, oils and all oil soluble or oil dispersible materials that may include liquids and solids such as stearic acid, zinc stearate, magnesium stearate, polyvinyl alcohol, polyvinylpyrollidone, carnauba wax, beeswax, microcrystralline wax, glyceryl stearate, glycol stearate, cetyl alcohol, stearyl alcohol, cetearyl alcohol, Isododecane, Diisocetyl Dodecaneodioate, Isohexadecane, Tocopherol, sunflower seed oil, meadofoam seed oil, hydrogenated meadofoam seed oil, polyisobutene, hydrogenated polyisobutene, paraffin, VP/Eicosene Copolymer. A phase is a group of ingredients that can be preblended or added in a given order at certain point in the process. A formula can have as many phases as needed. In most embodiments there are two main phases wherein one phase contains water and water soluble or dispersible ingredients, the other phase contains oils silicones and oil soluble or dispersible ingredients. Additional phases may be included, and when included generally are for the purpose of formula stability.

For example, in the example embodiment A there are 4 phases Phase A is water phase and is blended together, phase B is a pigment and is added after phase A is premixed. Phase C is an oil phase which is premixed and heated to a certain temperature. Phase D is a group of ingredients that is added in the given order to the premix of phases A, B, and C. The water phase and oil phase are first prepared separately. Both phases are heated separately to about 70-80 degrees Celsius (° C.). Then the water phase and oil phase are mixed together with continuous mixing. This mixture is cooled while mixing continues. Heat sensitive ingredients, (which include herbal extracts, such as Panax Ginseng Root Extract, Vaccinium Myrtillus Extract, Grapefruit extract, Rosemary leaf extract, Aloe vera gel, Acacia Senegal Flower/Stem Extract, Abies Koreana Leaf Extract, Acer Palmatum Leaf Extract, Aconitum Ciliare Root Extract, Acorus Calamus Rhizome Extract, Backhousia Anisata Leaf Extract, Bambusa Arundinacea Leaf Extract, Beta Vulgaris (Beet) Extract, Brassica Alba Seed Extract, Calendula Officinalis Extract, Camellia Japonica Flower Extract, Camellia Sinensis Leaf Extract, Canna Indica Root Extract, Carthamus Tinctorius (Safflower) Flower Extract, Chamomilla Vulgaris Extract, Cinnamomum Japonicum Leaf Extract, Citrus Aurantium Dulcis (Orange) Seed Extract, Coccinia Indica Fruit Extract, Coleus Forskohlii Root Extract, Echinacea Angustifolia Extract, Emblica Officinalis Fruit Extract, Garcinia Cambogia Fruit Extract, Glycyrrhiza Glabra (Licorice) Root Extract, Helianthus Annuus (Sunflower) Flower Extract, Hibiscus Abelmoschus Extract, Lavandula Angustifolia (Lavender) Extract, ycium Chinense Fruit Extract, Malus Domestica Fruit Cell Culture Extract, Melilotus Officinalis Flower Extract, Morus Alba Leaf Extract, a Europaea (Olive) Fruit Extract, Oryza Sativa (Rice) Bran Extract, Paeonia Suffruticosa Bark Extract, Panax Ginseng Root Extract, Pisum Sativum (Pea) Extract, Pueraria Lobata Root Extract, Punica Granatum Fruit Extract, Rehmannia Chinensis Root Extract, Rosa Alba Leaf/Stem Extract, Rosa Multiflora Root Extract, Sambucus Nigra Flower Extract, Saxifraga Sarmentosa Extract, Scutellaria Baicalensis Extract, Serenoa Serrulata Fruit Extract, Simmondsia Chinensis (Jojoba) Seed Extract, Swertia Japonica Extract, Tilia Cordata Flower Extract, Trifolium Pratense (Clover) Flower Extract, Triticum Vulgare (Wheat) Germ Extract, Tussilago Farfara (Coltsfoot) Flower/Leaf Extract, Veronica Officinalis Extract, Vitis Vinifera (Grape) Seed Extract, Zingiber Officinale (Ginger) Root Extract), are added when the mixture is at about 40° C. or below 40° C. The mixture, which may now also comprise heat sensitive ingredients, is mixed until it cools to room temperature (about 16° C. to 26° C.). A mascara in this disclosure will generally comprise water, and one or more of components designed to function as a thickener, a pH buffer, a humectant, a pigment, an emulsifier, a consistency factor, a film former, a water proofing agent, and a preservative.

A thickener is used to create the viscosity or consistency of the mascara so that it is suitable for the applicator to be used. Two types of thickeners are generally used depending upon whether the formulation is oil soluble and water soluble. The oil soluble thickener includes waxes such as beeswax, carnauba wax, ozokerite wax, micro crystalline wax, long chain (C16-C22) alcohols such as Cetyl, Stearyl or behenyl alcohol, Stearic Acid, magnesium stearate, Zinc Stearate, Hectorite clays such as Distrearyldimonium chloride (Bentone 38V), quaternium 18 hectorite (Lucentite San-P). The water soluble thickeners may include cellulosic thickeners (hydroxyethylcellulose), xanthan gum (keltrol), starch based thickeners such as Zea Mays (corn) starch, and Acrylate thickeners such as carbopols.

A pH buffer brings the pH of the product to the desired pH level. The pH is a measure of acidity/ alkalinity of the product. pH is measured with a pH meter. Mascaras are usually formulated at a pH level of 7 to 7.5, which is the desired pH level for the disclosed product. The pH buffer also maintains a product's pH, and may include a weak acid such as citric acid and alkaline ingredients such as triethanolamine, aminmomethyl propanol, sodium hydroxide, potassium hydroxide.

A humectant maintains moisture in the mascara and may include glycols (such as propylene, butylene, and pentylene glycols) and Glycerine.

A pigment provides color to the mascara. Generally the pigment is carbon black or an iron oxide.

An emulsifier is an ingredient or combination of ingredients used to create an emulsion. Emulsifiers are surfactants that modify the surface properties of other ingredients. Emulsifiers can be primary or secondary. Depending upon the formulation of the mascara, in some cases only a primary emulsifier is used to create a stable emulsion. In other cases both primary and secondary emulsifiers are needed to stabilize the emulsions. Emulsifiers may include Glyceryl Stearate, PEG 100 stearate, PEG-75 Stearate, Ceteth 20, Steaqreth 20, cetyl alcohol, stearyl alcohol.

A consistency factor is a component that provides viscosity/texture to the disclosed product. It is similar to thickener and is measured in terms of viscosity and may include any of the ingredients used for thickening the product, such as natural gums, for example Acacia gum, Hydroxyethyl cellulose, Hydroxymethylpropyl cellulose, corn starch, polymeric thickeners such as carbopols(carbomers).

A film former is used to allow for flexible lash-styling and shape-holding. A film former is generally a synthetic polymer such as Polyvinylpyrrolidone (PVP), and acrylic, styrene polymers and their derivatives. For example, useful film formers include polyvinyl pyrollidone/vinyl pyrollidone Eicosense Copolymer (Ganex V220), Isododecane & Butylene/Ethylene/Propylene Copolymer, Polyvinyl Acetate, Ethylene/VA Copolymer, Acrylates Copolymer, Acrylates/Hydroxyesters Acrylates Copolymer, and PVP/Dimethylaminoethyl Methacrylate Copolymer.

A waterproofing agent is obtained by a combination of wax and a suitable film forming polymer. For example a polymer such as Ganex V220 will provide waterproofing to the mascara. Other waterproofing polymers may include trimethylpentanediol/adipic acid/glycerin crosspolymer (marketed commercially under the name Lexorez 200), isododecane and butylene/ethylene/propylene copolymer.

A preservative protects the mascara from contaminants and keeps it fresh during the mascara's shelf life and use life. A preservative may include glycols such as propylene glycol, butylene glycol, pentylene glycol, caprylyl glycol, ethyl alcohol, benzyl alcohol, phenoxyethanol, ethylhexyl glycerin, methylparaben, propylparaben, butylparaben, ethylparaben, imidazolidinyl urea.

Below is described a particular embodiment of a mascara of this disclosure having 4 phases.

In this embodiment, Phase A is water soluble phase and is premixed, Phase B is a pigment and is added to the premixed phase A, Phase C is oil phase and is premixed separately and then mixed in to the phase AB, and Phase D is a group of ingredients that are added to the mixture of phase ABC in the given order.

### Mascara Embodiment A

| | | Amount |
|---|---|---|
| Ingredient | Purpose | % by wt. |
| Phase A | | |
| Water | | Q.S. |
| Hydroethylcellulose | Thickener | 0.5 |
| Triethanolamine | pH Buffer/Neutralizer | 2.0 |
| Butylene glycol | Humectant | 8.0 |
| Phase B | | |
| Iron oxides (for example | Pigment | 10.0 |
| INCI CI 77499, which is a | | |
| Triiron tetraoxide) | | |
| Phase C | | |
| Glyceryl stearate | Emusifier | 2.5 |
| Carnauba Wax | Consistency factor | 4.0 |
| Beeswax | Consistency factor | 5.0 |
| Candelilla Wax | Consistency factor | 1.0 |
| Stearic acid | Emulsifier | 5.0 |
| Phase D | | |
| Acrylates copolymer | Film former | 5.0 |
| Dimethicone | Waterproofing agent | 0.5 |
| Phenoxyethanol, | Preservative | 0.7 |
| methylparaben, | | |
| ethylparaben, | | |
| propylparaben, | | |
| butylparaben, | | |
| isobutylparaben | | |
| ethylhexyl glycerin, | | |
| Caprylyl Glycol | | |

A transplanting gel in this disclosure is a gel type mascara. In a specific example a transplanting gel comprises the ingredients in the chart above. In another embodiment a transplanting gel comprises water, beeswax (natural propolis), carnauba (Brazilian palm glue), iron oxide cl 77499 (black), collagen, acrylates copolymer, nylon, stearic acid, and propylene glycol.

In a further specific embodiment the transplanting gel comprises:

### Transplanting Gel Embodiment A

| | Amount (% by Weight of |
|---|---|
| Ingredient | the Transplanting Gel) |
| Water | 49.8700 |
| Butylene Glycol | 9.0000 |
| Iron Oxides (CI 77499) | 9.0000 |
| Acrylates Copolymer | 8.4000 |
| Stearic Acid | 6.3000 |
| Polyvinly Alcohol | 3.0000 |
| Copernicia Cerifera (Carnauba) Wax | 2.7000 |
| Tromethamine | 2.5200 |
| Beeswax | 1.8000 |
| Silica | 1.0000 |
| Hydroxyethylcellulose | 1.0000 |
| Microcrystalline Wax | 0.9000 |
| Glyceryl Stearate | 0.9000 |
| Cetearyl Alcohol | 0.9000 |
| Alcohol | 0.8000 |
| Allantoin | 0.5000 |
| Dimethicone | 0.4500 |
| Phenoxyethanol | 0.3725 |
| PPG-2-Deceth-30 | 0.3200 |
| Ethylhexyl Glycerine | 0.1275 |
| Sodium Laureth Sulfate | 0.0800 |
| Sodium Dehydroacetate | 0.0600 |

A fiber in this disclosure is a rayon fiber. A rayon fiber is a cellulose based fiber, which enhances its appeal to environmentally conscious consumers and also allows it to be dyed in a wide range of colors. A rayon fiber can be made by the acetate method, cuprammonium method, or the viscose method, though the viscose method is believed to result in particularly useful fibers. Examples of commercially available rayon fibers are offered under the trade names Viscose, Lyocell, and Modal.

A fiber has the characteristics of thickness, length, configuration, surface treatment, and color.

Fiber thickness is measured in denier, which is a measure for the linear mass density of fibers. In this disclosure, denier means the average denier of the fibers used. Fiber thicknesses for the present disclosure is 0.2 to 12 denier, though a specific thickness that may be used is 1 to 8 denier, 1.5 to 6 denier, 3 to 4 denier, such as 3 denier, and even more particularly, 0.4 -0.5 denier.

Fiber length is measured along the longitudinal axis of the fiber. Fibers with a length ranging from 0.1 millimeters (mm) to 8 mm can be used in the present disclosure, though in particular embodiment's fibers with lengths from 0.05 mm to 6 mm, 1 mm to 3 mm, or 1.5 mm to 2 mm are used. In a particular embodiment of this disclosure the fiber used is 2 mm long and 3 denier thick

Fiber configuration is whether the fiber is generally round or generally angular and generally straight or generally curved. In specific embodiments, generally round fibers are used. Though, in certain embodiments the cross section of the fiber can be round, triangular or star shaped (or a combination of these cross sections).

The fibers of the present disclosure may be treated to create or enhance three or more different characteristics.

First, the fibers may be treated so that they have a certain color. Colors include black, natural, or any other color. In some cases, the fiber is treated to match the color of the mascara. This is a great advancement in this technology as normally fibers are not colored and may be a different color from the mascara. In other cases, the fiber is treated with any color, which may be similar to the mascara color, or not match the mascara color. In certain examples the fibers are treated with carbon black so that they will have a very dark and/or black coloration. Iron oxides may also be used to obtain a black color. For other colors common fabric dyes approved internationally for dyeing fabrics can be used. In certain cases FD&C colors can also be used to color the fibers.

Second, the fibers may be treated to condition the fibers. Conditioning of a fiber may be useful to reduce static charge and flyaway. Exemplary conditioners used in the present disclosure are of cationic type and can belong to many different classes. These conditioners are commonly used in fabric softener or hair conditioning technology. A tertiary alkyl ammonium chloride type conditioner can be used at low levels ranging from 0.5 to 3% by weight to condition the fibers making them perform better in the disclosed method. This conditioning may be performed to reduce the static electricity of the fiber to reduce the tendency of the fiber to detach from the brush during application to a subject's eyelash, detach from the subject's eyelash after application, and to make the fiber easier to apply to a subject's eyelash.

Third, the fiber may be treated so as to allow the composition to calm, sooth, and nourish a user's eyelashes. In some examples, the fiber may be treated with antioxidants such as aloe vera, grapeseed extract, pomegranate seed extract, goji berry extract, centella asiatica extract, emblica extract, rosemary extract, ginseng extract, green tea extract, or water soluble vitamins and their derivatives such as dl-panthenol, Biotin and others of the B series, ascorbic acid, ethyl ascorbate, or any other antioxidant which would be tolerated by a user, such that the fiber comprises an antioxidant, by virtue of being coated with or impregnated with the antioxidant.

The following is an example of an embodiment of a process of making the disclosed fiber for the disclosed cosmetic composition. In this exemplary embodiment:
1. Long natural color rayon fibers are colored black with carbon black, iron oxide or any other black dyes such as aniline black. In this particular example carbon black is used. These long colored rayon fibers are called tow.
2. The tow is then cut for cosmetic purposes to range of 0.5 to 4 mm. In this case embodiment, the tow is cut to an average of 2 mm. These cut fibers are now called flock.
3. The flock is treated with one or more conditioners to reduce static and flyaway and enhance manageability of the end fiber.
4. The flock is then treated with an antioxidant herbal extract such as Aloe vera, Iris Pallida leaf cell extract, ginseng extract, green tea extract, or grape seed extract to calm, sooth, and/or nourish a user's eyelashes. For the same purpose it can also be treated with water soluble vitamins and their derivatives such as dl-panthenol, Biotin and others of the B series, ascorbic acid, and ethyl ascorbate.
5. To further enhance manageability of the fiber, it is treated with a lipophilic material. The lipophilic material includes any lipophilic material that will be tolerated by a user and can be an oil such as argan oil, sunflower oil, grape seed oil, squalene, capryllic/capric triglyceride, linoleic acid, borage oil, an ester such as isopropyl myristate, ceteary octanoate, C12-15 Alkyl Benzoate (or any liquid esters), liquid silicones, and mineral oils. The lipophilic material can also be a blend of oils, or blend of oils, esters and oil soluble vitamins such as tocopherol or tocopherol acetate, tetrahexyldecyl ascorbate, Coenzyme Q10, Ascorbyl Palmitate. It can also be a blend of oils, esters, vitamins, mineral oil and silicones. In a particular case, such as this one, the lipoplhilic material is Squalane, C12-15 Alkyl Benzoate, Tetrahexyldecyl Ascorbate, Tocopheryl acetate,

Coenzyme Q10, Iris Pallida Leaf Cell Extract, and capryllic/capric triglycerides. The quantity of oil or blend depends upon the type of oil or the blend. Generally it might range from 0.1-20% by weight of the dry fibers, 1-15% by weight of the dry fibers, or from 4 -10% or 5-9% by weight of the dry fibers. In this particular example, the quantity of oil or blend ranges from 6-8% by weight of the dry fibers.

The disclosed method of lengthening the appearance of a subject's eye lashes comprises applying mascara and/or transplanting gel to a subject's eyelashes and then applying the disclosed fiber to the subject's eyelashes. This is accomplished by applying the mascara and/or transplanting gel to the subject's eyelashes with a brush and then applying the fiber to subject's eye lashes with a separate brush. This is generally accomplished by having the mascara and/or transplanting gel in one or two containers and the fiber in a separate, second or third container, each having its own brush. The first brush is for the mascara or transplanting gel and is dipped into the mascara and/or transplanting gel container to cause the bristles of the brush to become coated with mascara and/or transplanting gel. The brush is then use to apply the mascara and/or transplanting gel to the subject's eyelashes. Once the mascara and/or transplanting gel has been applied, the second brush is dipped into the fiber container so that the fibers temporarily attach to the brush. The brush with the fiber is then used to apply the fibers to the subject's eyelashes, by, for example, stroking the brush along the subject's eyelashes from the root of the eyelash (the portion nearest the eyelid) to the tip of the eyelash (the portion farthest away from the eyelid). The mascara and/or transplanting gel causes the fibers to transfer from the brush to the mascara and/or transplanting gel coated eyelashes as the brush strokes along the eyelash. The directional nature of the brush strokes assist the fibers to align in a substantially parallel fashion with the subject's eyelashes.

In embodiments in which transplanting gel is applied first to assist with application of the fibers, mascara may then optionally be applied over the transplanting gel/fiber combination in the same manner as the transplanting gel was applied.

Disclosed systems include two or more containers comprising in the first container, the fiber, and in two container systems, the mascara, with the transplanting gel in a second container. In a three container system the first container holds the fiber, the second container holds the transplanting gel, and the third container holds the mascara. The systems can be used in the methods described above.

The following embodiments of the disclosed method and system illustrate use of the method to enhance the appearance of eyelash length according to the present disclosure.

### Embodiment 1

1. The transplanting gel (black in color) is first applied from a container to a user's eyelashes with a brush gently from the back of eyelashes to the front in order to coat the eyelashes evenly.
2. Fibers treated with aloe vera and vitamins C, E & coenzyme Q10 are then applied from another container with the help of a second brush. The function of the brush is to easily release the fibers to the eyelashes coated with the gel that has not completely dried.
3. In some cases another coat of the transplanting gel may be applied to hold the fibers in place.
4. The mascara is then applied from another container over the transplanting gel and fiber.

### Embodiment 2

1. The transplanting gel is applied as described above in embodiment 1.
2. In this embodiment the fibers are treated differently to achieve different results. The level of treatment of fibers with the oils or oil blends can produce different results. The fibers that are under treated with oils may produce frizzy eyelashes or lashes with higher volume in addition to the length. In this embodiment the fibers are treated to make the fibers more manageable and reduce flyaway. This is accomplished by treating the fibers with an anhydrous liquid such as a natural oil or blend of oils such as silicone oil and mineral oil. In some cases, the fibers are also treated with oil soluble herbal extracts such as Rhatany Root Extract, Ginger extract, Chamomile Extract (croda), Aloe vera gel oil soluble. The fibers are treated with the oil in a sufficient amount to make the fibers more manageable and reduce flyaway but not so much that the fibers feel oily to the touch. The process of treating the fibers is to add the anhydrous liquid very slowly to the fibers during mixing in a Hobart or Hobart type planetary mixer for 15-30 minutes. The fibers are then transferred to another type of mixer called a ribbon blender to make sure that the treatment ingredient is distributed uniformly throughout the fibers. Fibers treated in this manner do not produce the frizzy effect and only extend the eye lashes. If the fibers are over treated with the oils or oil blends they do not easily transfer to the eyelashes. Overtreatment can be recognized by feeling the fibers. They are over treated if the fibers feel oily to the touch.

### Embodiment 3

1. The transplanting gel is applied as described above in embodiment 1.
2. Fibers are applied to the user's eyelash and transplanting gel that are of the same color as the gel.
3. Mascara is then applied over the transplanting gel and fiber.

### Embodiment 4

1. The transplanting gel (black in color) is first applied from a container to a user's eyelashes with a brush gently from the back of eyelashes to the front in order to coat the eyelashes evenly.
2. Fibers treated with aloe vera which are combined with mascara are then applied from another container with the help of a second brush. The function of the brush is to easily release the fibers to the eyelashes coated with the gel that has not completely dried.

In view of the above and many possible additional embodiments to which the principles of the present disclosure may be applied, it should be recognized that the illustrated embodiments are only exemplary and should not be taken as limiting the scope of the disclosure. The scope of the disclosure is defined by the following claims. I therefore claim as my disclosure all that comes within the scope and spirit of these claims.

I claim:

1. A method of enhancing the apparent length of a subject's eyelashes comprising:
Applying to the subject's eyelashes a transplanting gel from a first container,
Applying to the subject's eyelashes a fiber from a second container.

2. The method of claim 1, wherein:
A mascara is applied to the subject's eyelashes.

3. The method of claim 1, wherein:
The fiber comprises cellulose.

4. The method of claim 3, wherein:
The fiber comprises rayon.

5. The method of claim 3, wherein the fiber has a length of from 1mm to 3mm.

6. The method of claim 5, wherein the fiber has a length of from 1.5 mm to 2 mm.

7. The method of claim 3, wherein the fiber has a denier of 2-4.

8. The method of claim 7, wherein the fiber has a denier of 3.

9. The method of claim 3, wherein the fiber is conditioned.

10. The method of claim 9, wherein the fiber is conditioned with a tertiary alkyl ammonium chloride.

11. The method of claim 3, wherein the fiber comprises an antioxidant.

12. The method of claim 11, wherein the fiber comprises aloe vera.

13. The method of claim 11, wherein the fiber comprises aloe vera, vitamin C, and coenzyme Q10.

14. The method of claim 3, wherein the fiber and the transplanting gel have the same color.

15. The method of claim 1, wherein the transplanting gel comprises:

| | % by weight |
|---|---|
| Water | 49.8700 |
| Butylene Glycol | 9.0000 |
| Iron Oxides (CI 77499) | 9.0000 |
| Acrylates Copolymer | 8.4000 |
| Stearic Acid | 6.3000 |
| Polyvinly Alcohol | 3.0000 |
| Copernicia Cerifera (Carnauba) Wax | 2.7000 |
| Tromethamine | 2.5200 |
| Beeswax | 1.8000 |
| Silica | 1.0000 |
| Hydroxyethylcellulose | 1.0000 |
| Microcrystalline Wax | 0.9000 |
| Glyceryl Stearate | 0.9000 |
| Cetearyl Alcohol | 0.9000 |
| Alcohol | 0.8000 |
| Allantoin | 0.5000 |
| Dimethicone | 0.4500 |
| Phenoxyethanol | 0.3725 |
| PPG-2-Deceth-30 | 0.3200 |
| Ethylhexyl Glycerine | 0.1275 |
| Sodium Laureth Sulfate | 0.0800 |
| Sodium Dehydroacetate | 0.0600 |

16. The method of claim 15, wherein the fiber comprises rayon, is colored black and comprises aloe vera.

17. A method of enhancing the apparent length of a subject's eyelashes comprising:
Applying to the subject's eyelashes a transplanting gel from a first container, wherein the transplanting gel comprises:

| | % by weight |
|---|---|
| Water | 49.8700 |
| Butylene Glycol | 9.0000 |
| Iron Oxides (CI 77499) | 9.0000 |
| Acrylates Copolymer | 8.4000 |
| Stearic Acid | 6.3000 |
| Polyvinly Alcohol | 3.0000 |
| Copernicia Cerifera (Carnauba) Wax | 2.7000 |
| Tromethamine | 2.5200 |
| Beeswax | 1.8000 |
| Silica | 1.0000 |
| Hydroxyethylcellulose | 1.0000 |
| Microcrystalline Wax | 0.9000 |
| Glyceryl Stearate | 0.9000 |
| Cetearyl Alcohol | 0.9000 |
| Alcohol | 0.8000 |
| Allantoin | 0.5000 |
| Dimethicone | 0.4500 |
| Phenoxyethanol | 0.3725 |
| PPG-2-Deceth-30 | 0.3200 |
| Ethylhexyl Glycerine | 0.1275 |
| Sodium Laureth Sulfate | 0.0800 |
| Sodium Dehydroacetate | 0.0600 |

Applying to the subject's eyelashes a fiber from a second container wherein the fiber comprises rayon and an antioxidant selected from the group of aloe vera, grapeseed extract, pomegranate seed extract, goji berry extract, centella asiatica extract, emblica extract, rosemary extract, ginseng extract, green tea extract, has a denier of 3, and is conditioned.

18. A system for enhancing the apparent length of a subject's eyelashes comprising:
A first container having therein a transplanting gel;
A second container having therein a fiber comprising rayon and an antioxidant;
A brush for applying the transplanting gel and a brush for applying the fiber.

19. The system of claim 18 wherein the fiber has a denier of 3.

20. The system of claim 18 wherein the antioxidant is selected from the group of aloe vera, grapeseed extract, pomegranate seed extract, goji berry extract, centella asiatica extract, emblica extract, rosemary extract, ginseng extract, green tea extract.
